(19) United States
(12) Patent Application Publication
(10) Pub. No.: US 2010/0305515 A1
(43) Pub. Date: Dec. 2, 2010

(54) Locking Mechanism for Drug Delivery System

(76) Inventors: Veerappan S. Subramanian, Mendham, NJ (US); Muthusamy Shanmugan, Monmouth Junction, NJ (US); Yuh-Herng Eric Lin, East Brunswick, NJ (US); Michael Basil, East Hampton, CT (US)

Correspondence Address: Barbara V. Maurer, 5 Haven Hill Rise, Flemington, NJ, 08822, US

(21) Appl. No.: 12/789,373

(22) Filed: May 27, 2010

Related U.S. Application Data
(60) Provisional application No. 61/217,196, filed on May 28, 2009.

(51) Int. Cl. A61M 5/315

(52) U.S. Cl. 604/246

(57) ABSTRACT

The invention relates to a locking mechanism for adjustable systems for delivering a specified dosage of a substance. The delivery system can be used to deliver, for example, a drug or a nutritional supplement to a human or veterinary patient or for delivering, for example, fertilizer in agricultural use.

This application claims priority of prior U.S. provisional application Ser. No 61/217,196, filed May 28, 2009, incorporated herein by reference in its entirety.

The present invention relates to a locking mechanism for adjustable systems for delivering a specified dosage of a substance. The delivery system can be used to deliver, for example, a drug or a nutritional supplement to a human or veterinary patient or for delivering, for example, fertilizer in agricultural use.

Often different amounts of a substance must be administered depending on the situation. For example, all human or veterinary patients do not often receive identical doses of drugs. Factors that influence a difference in drug dosage can include, for example, the size and weight of the patient, the age of the patient, the sex of the patient, whether the patient is a young or an adult, etc. Currently, in order to administer the proper dose to the patient, drug delivery devices typically require that the device is manufactured such that the entire volume is metered to deliver the desired dose of the drug, or at the point of care a provider must adjust the volume to be delivered, thereby adjusting the dose themselves. Both of these options, however, have certain inherent drawbacks.

In one case, the manufacturer is required to make several versions of a device, with each version including a different volume (usually incremental) of the drug. Further, because several different versions of the device are required, both the distributor of the drug and the pharmacist are required to use more “shelf space” by keeping an inventory of the various devices containing different amounts of the drug. The same type of problems hold true for substances such as fertilizers for agricultural use.

In the case where a provider adjusts the dosage, it is more likely (as compared to a pharmacist) that the provider might make an error in setting the proper dosage. This could be especially important if the patient requires an immediate administration of a drug.

Thus there remains a need for a delivery system that can be adjusted at a suitable dispensing location such as a pharmacy, nursing station, or agricultural station.

Accordingly, an objective of the instant invention is to provide for a device having an adjustable mechanism for determining dosage so that a dispenser or other provider is not obligated to stock several delivery devices having different dosages.

Another objective of the invention is to provide a drug delivery system having a locking system so that the desired dosage can be set by a dispensing specialist, for example a pharmacist or agricultural specialist, thereby decreasing the risk of dispensing an undesired dosage.

A locking system for a typical syringe-type drug delivery system has now been developed that meets the objectives set forth above. In this system, a locking ring is positioned around the plunger of the syringe between the barrel of the syringe and the end of the plunger. In use, the plunger of the system is positioned and then locked at the point necessary for the delivery of the desired amount of the substance. The locking mechanism is applicable to drug delivery devices suitable for subcutaneous, intramuscular, oral, buccal, otic, nasal, topical or rectal use in humans and veterinary patients, or for delivery into the soil or a delivery medium such as water for spraying.

The invention is more fully understood with the aid of the description below and the accompanying figures wherein:

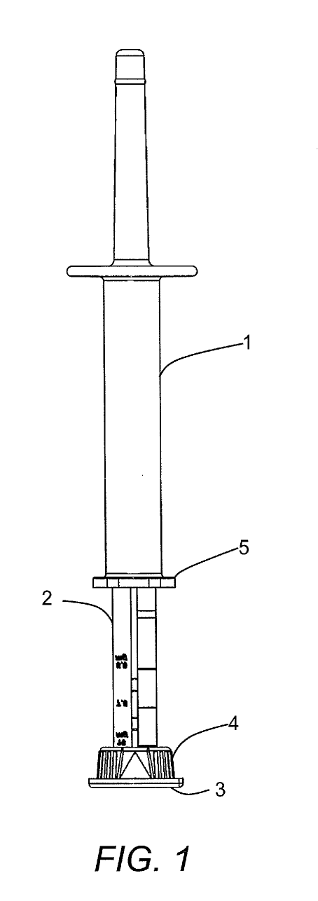

FIG. 1 shows the assembled drug delivery system of the invention;

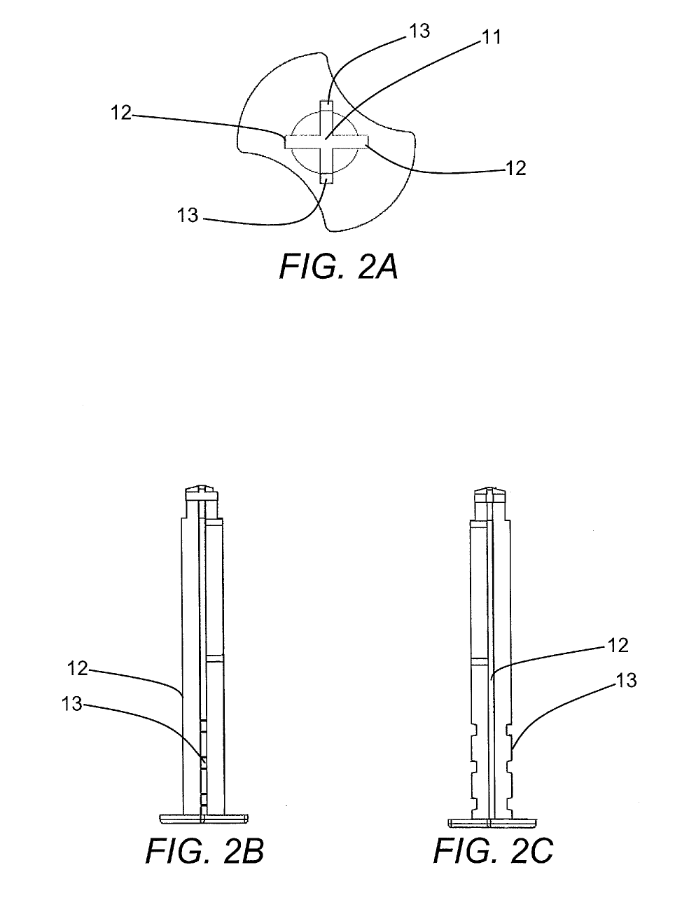

FIG. 2 shows multiple views of the plunger rod wherein A shows a cross-section, and B and C show a linear view of the plunger rod; and

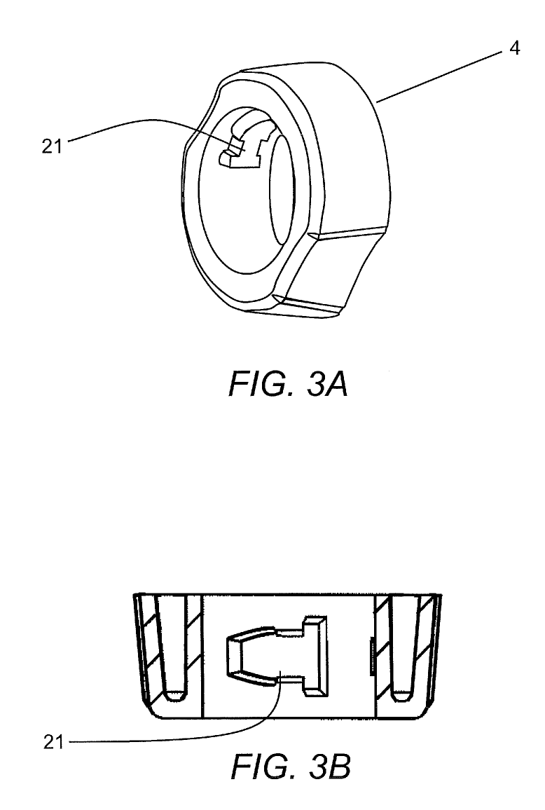

FIG. 3 shows the dial ring of the invention.

More particularly, a substance is delivered in the simple delivery system of the invention that can be preset with the desired dose. The plunger has a saw tooth design which, with the dial ring, forms the locking mechanism. The plunger saw tooth design is calibrated with dosage strength stamped on it. The dial ring can be positioned and locked at the desired dosage marked on the plunger resulting in the delivery device delivering an accurate dose. The dial ring can be grooved to enhance the grip. The dial ring optionally has an arrow which indicates the dosage strength. The syringe when locked at any strength results in the exact desired amount of substance to be delivered. The locking system is simple and user friendly.

Reference is now made to FIGS. 1-3, which illustrate pictorially various embodiments of the invention.

The assembled drug delivery system of the invention is shown in FIG. 1 wherein 1 is the barrel of a syringe having a plunger 2 inserted therein. A dial ring 4 is positioned on the plunger 2 between the end of the barrel 5 and the end of the plunger rod 3.

FIG. 2 shows multiple views of the plunger 2. FIG. 2A shows a cross section of the plunger where it can be seen that the plunger has a center core 11 and four flanges that run the length of the plunger. Two of the flanges 12 have straight edges and the remaining two flanges 13 have notched edges. As can be seen in FIG. 2B the flanges 12 are inscribed with dosage amounts and flanges 13 have notched edges that correspond to the dosage markings on flanges 12. FIG. 2C shows the plunger rotated 90° .

FIG. 3A shows the dial ring 4 of the invention, having a locking configuration 21 on the inside diameter of the ring. FIG. 3B shows a cross-section of the dial ring showing the locking mechanism 21. The ring can slide freely along the axis of the plunger.

In practice, the dial ring 4 can fit against the inner edge of the plunger end 3 until it is used to lock a desired dosage. A particular dosage is set by sliding the dial ring to the appropriate marking on the plunger flange 12 and then twisting the dial ring until it engages with a notch on the notched flanges 13 of the plunger.

In use, the dial ring is irreversibly locked when it is twisted to lock at a particular dosage.

It is contemplated that the delivery system of the invention can be used in many applications. For delivery to a human or animal, the administration can be rectal, oral, topical, buccal, or parenteral. Parenteral administration includes subcutaneous, intravenous, intramuscular and intrasternal administration. The device can be used with any substance that is preferentially administered as a single dose, as is known in the art. Examples include insulin, ointments on the skin, infant sized dosages of liquid medications, vaccines, and the like.

Especially in the veterinary field, it can also be used to give a dose of liquid injected subcutaneously with a hypodermic needle, such as saline solution administered either to counteract dehydration or especially to mitigate kidney failure, a common ailment in domestic cats.

In agricultural use, the delivery system can be used to deliver a predetermined amount of fertilizer, herbicide or fungicide either to the target directly or to a medium used to deliver the substance to the individual plant or field of crop as is deemed necessary.

An example of a drug delivery system of the invention is for the treatment of patients who experience epileptic seizures. Typically, patients who suffer increased and intermittent seizure activity due to epilepsy are treated via a rectal drug delivery device that administers a pre-set dose of a drug such as diazepam in a gel form. As discussed above, there is a need for a system that employs a conventional syringe for delivering an adjustable pre-set dosage of a drug. Accordingly, the delivery system of the instant invention can be used for this purpose.

The systems and methods of the present invention may be embodied in other specific forms without departing from the teachings or essential characteristics of the invention. The described embodiments are therefore to be considered in all respects as illustrative and not restrictive, the scope of the invention being indicated by the appended claims rather than by the foregoing description, and all changes which come within the meaning and range of equivalency of the claims are therefore to be embraced therein.

1. A substance delivery system comprising
a) a syringe having an outer barrel and a plunger moveably inserted within the barrel; and
b) a slidable and lockable dial ring mounted on the plunger;
wherein the dial ring can be locked on the plunger.

2. The system of claim 1 wherein the dial ring is rotated to lock on the plunger.

3. The system of claim 2, wherein the plunger further comprises a dosage indicator.

4. The system of claim 3, wherein the dosage indicator provides a predetermined number of dosages of the substance.

5. The system of claim 1 which is adapted for human use.

6. The system of claim 1 which is adapted for veterinary use.

7. The system of claim 1 which is adapted for agricultural use.
